(19) United States
(12) Patent Application Publication
(10) Pub. No.: US 2013/0162945 A1
(43) Pub. Date: Jun. 27, 2013

(54) OPHTHALMOLOGIC APPARATUS, AND OPHTHALMOLOGIC CONTROL METHOD AND PROGRAM

(71) Applicant: CANON KABUSHIKI KAISHA, Tokyo (JP)

(72) Inventor: Ryuji Tanaami, Yokohama-shi (JP)

(73) Assignee: CANON KABUSHIKI KAISHA, Tokyo (JP)

(21) Appl. No.: 13/710,815

(22) Filed: Dec. 11, 2012

(30) Foreign Application Priority Data
Dec. 21, 2011 (JP) 2011-279585

(51) Int. Cl. A61B 3/12; A61B 3/14

(52) U.S. Cl. CPC A61B 3/12; A61B 3/14
USPC 351/206

(57) ABSTRACT

Provided is an ophthalmologic apparatus, which can easily and quickly find a part of a crystalline lens without opacity where specific information of an eye to be inspected can be acquired (for example, eye refractive power information can be measured), when changing to a transillumination observation mode. The ophthalmologic apparatus includes: a specific information acquiring unit which acquires specific information of the eye to be inspected through a first opening; a transillumination image acquiring unit which acquires a transillumination image of the eye to be inspected; and a control unit which changes the first opening to a second opening smaller than the first opening when acquiring the transillumination image.

### BACKGROUND OF THE INVENTION

1. Field of the Invention

The present invention relates to an ophthalmologic apparatus, and an ophthalmologic control method and program, each having a function of acquiring specific information of an eye to be inspected (for example, measuring eye refractive power or the like of the eye to be inspected) and a function of observing an illuminated pupil area by a reflected light beam from a fundus of the eye to be inspected (transillumination observation).

2. Description of the Related Art

As to an ophthalmologic apparatus for measuring eye refractive power or the like of an eye to be inspected, the following structure is known as disclosed in Japanese Patent No. 4233426. When measuring the eye to be inspected having a small pupil diameter, for example, of a senior person, it is automatically determined whether or not a measuring beam projected to the fundus via a ring type opening disposed at a position conjugate with the pupil is vignetted by the iris of the eye to be inspected. Then, as a result of the automatic determination based on a measured image or an anterior eye part image, when it is determined that the measuring beam is vignetted, the diameter of the ring type opening is changed to be smaller so as to perform the measurement in a state where the measuring beam is not vignetted by the pupil.

However, the ophthalmologic apparatus of Japanese Patent No. 4233426 automatically determines whether or not the measuring beam is vignetted by the iris of the eye to be inspected, but the ring type opening is not automatically changed from one for a normal pupil diameter to one for the small pupil diameter when the crystalline lens is opacified. Therefore, when searching for a measurable part of the crystalline lens without opacity by transillumination observation, the ring type opening for the normal pupil diameter is disposed on the optical axis so that the measuring beam diameter is large. As a result, when the opacified area of the crystalline lens is large, a ring image for the normal pupil diameter is vignetted. When the transillumination observation is performed in the vignetted state in this way, a measurable part may not be found.

### SUMMARY OF THE INVENTION

It is an object of the present invention to provide an ophthalmologic apparatus, which can easily and quickly find a part of a crystalline lens without opacity where specific information of an eye to be inspected can be acquired (for example, eye refractive power information can be measured), by automatically changing an opening conjugate with the pupil of the eye to be inspected to an opening having a smaller radius from an optical axis in at least three meridional directions, when changing to a transillumination observation mode.

In addition, it is another object of the present invention to provide an ophthalmologic apparatus, which can easily and quickly acquire specific information at the part of the crystalline lens without opacity where specific information of the eye to be inspected can be acquired (for example, eye refractive power information can be measured), by automatically changing the opening conjugate with the pupil of the eye to be inspected to an opening having a larger radius from the optical axis in at least three meridional directions, when changing to a specific information acquisition mode of the eye to be inspected (for example, an eye refractive power information measurement mode).

In order to achieve the above-mentioned object, according to an exemplary embodiment of the present invention, there is provided an ophthalmologic apparatus, including: a specific information acquiring unit which acquires specific information of an eye to be inspected through a first opening; a transillumination image acquiring unit which acquires a transillumination image of the eye to be inspected; and a control unit which changes the first opening to a second opening smaller than the first opening when acquiring the transillumination image.

Further, according to another exemplary embodiment of the present invention, there is provided an ophthalmologic apparatus, including: an eye refractive power information measuring unit which projects a light beam to a fundus of an eye to be inspected and photographs a reflected light beam from the fundus of the eye to be inspected so as to measure eye refractive power information, the eye refractive power information measuring unit including a first opening having an opening in at least three meridional directions at a position conjugate with a pupil of the eye to be inspected in one of a reflection optical path from the fundus of the eye to be inspected and a projection optical path to the fundus of the eye to be inspected; and a transillumination observation unit which observes a pupil area of the eye to be inspected illuminated by the reflected light beam from the fundus of the eye to be inspected. In a transillumination observation mode for performing transillumination observation, the ophthalmologic apparatus is capable of photographing the reflected light beam from the fundus of the eye to be inspected in at least three meridional directions through a second opening having an opening in the at least three meridional directions at the position conjugate with the pupil of the eye to be inspected in the one of the reflection optical path from the fundus of the eye to be inspected and the projection optical path to the fundus of the eye to be inspected, and is capable of changing an aligned state of the ophthalmologic apparatus with respect to the eye to be inspected based on a state of the photographed reflected light beam. The ophthalmologic apparatus further includes a control unit which automatically changes the first opening in an eye refractive power information measurement mode to the second opening in the transillumination observation mode when changing from the eye refractive power information measurement mode using the eye refractive power information measuring unit to the transillumination observation mode. A radius of the opening from an optical axis when the second opening is projected to the pupil of the eye to be inspected is set smaller than a radius of the opening from the optical axis when the first opening is projected to the pupil of the eye to be inspected.

Further, according to still another exemplary embodiment of the present invention, there is provided an ophthalmologic apparatus, including: an eye refractive power information measuring unit which projects a light beam to a fundus of an eye to be inspected and photographs a reflected light beam from the fundus of the eye to be inspected so as to measure eye refractive power information, the an eye refractive power information measuring unit including a first opening having an opening in at least three meridional directions at a position conjugate with a pupil of the eye to be inspected in one of a reflection optical path from the fundus of the eye to be inspected and a projection optical path to the fundus of the eye to be inspected; and a transillumination observation unit which observes a pupil area of the eye to be inspected illuminated by the reflected light beam from the fundus of the eye to be inspected. In a transillumination observation mode for performing transillumination observation, the ophthalmologic apparatus is capable of photographing the reflected light beam from the fundus of the eye to be inspected in at least three meridional directions through a second opening having an opening in the at least three meridional directions at the position conjugate with the pupil of the eye to be inspected in the one of the reflection optical path from the fundus of the eye to be inspected and the projection optical path to the fundus of the eye to be inspected, and is capable of changing an aligned state of the ophthalmologic apparatus with respect to the eye to be inspected based on a state of the photographed reflected light beam. The ophthalmologic apparatus further includes a control unit which automatically changes the second opening in the transillumination observation mode to the first opening in an eye refractive power information measurement mode when changing from the transillumination observation mode to the eye refractive power information measurement mode using the eye refractive power information measuring unit. A radius of the opening from an optical axis when the first opening is projected to the pupil of the eye to be inspected is set larger than a radius of the opening from the optical axis when the second opening is projected to the pupil of the eye to be inspected.

Further, according to an exemplary embodiment of the present invention, there is provided an ophthalmologic control method, including: a specific information acquiring step of acquiring specific information of an eye to be inspected through a first opening; a transillumination image acquiring step of acquiring a transillumination image of the eye to be inspected; and a control step of changing the first opening to a second opening smaller than the first opening when acquiring the transillumination image.

According to another exemplary embodiment of the present invention, there is provided an ophthalmologic control program.

According to the present invention, when changing to the transillumination observation mode, the opening conjugate with the pupil of the eye to be inspected is automatically changed to the opening having a smaller radius from the optical axis in the at least three meridional directions. Thus, it is possible to easily and quickly find the part of the crystalline lens without opacity where specific information of the eye to be inspected can be acquired (for example, the eye refractive power information can be measured). Further, when changing to the specific information acquisition mode of the eye to be inspected (for example, the eye refractive power information measurement mode), the opening conjugate with the pupil of the eye to be inspected is automatically changed to the opening having a larger radius from the optical axis in the at least three meridional directions. Thus, in the part of the crystalline lens without opacity where specific information of the eye to be inspected can be acquired (for example, the eye refractive power information can be measured), the specific information can be easily and quickly acquired.

Further features of the present invention will become apparent from the following description of exemplary embodiments with reference to the attached drawings.

### BRIEF DESCRIPTION OF THE DRAWINGS

FIG. 1 is a layout diagram of an optical system of a measurement unit of an eye refractometer according to an embodiment of the present invention.

FIG. 2 is an outside view of the eye refractometer according to the embodiment of the present invention.

FIG. 3 is a perspective view of an alignment prism stop of the measurement unit of the eye refractometer according to the embodiment of the present invention.

FIG. 4A is an explanatory diagram of an initial state of a diaphragm plate changing mechanism of the eye refractometer according to the embodiment of the present invention, and FIG. 4B is an explanatory diagram of a driven state of the diaphragm plate changing mechanism.

FIG. 5 is an enlarged detailed diagram of a ring type opening disposed to the diaphragm plate changing mechanism.

FIG. 6A is a transillumination image in a case where a crystalline lens is opacified, and FIG. 6B is an explanatory diagram of a ring image to be photographed.

FIG. 7 is a flowchart of the eye refractometer according to the embodiment of the present invention.

### DESCRIPTION OF THE EMBODIMENTS

Preferred embodiments of the present invention will now be described in detail in accordance with the accompanying drawings.

### First Embodiment

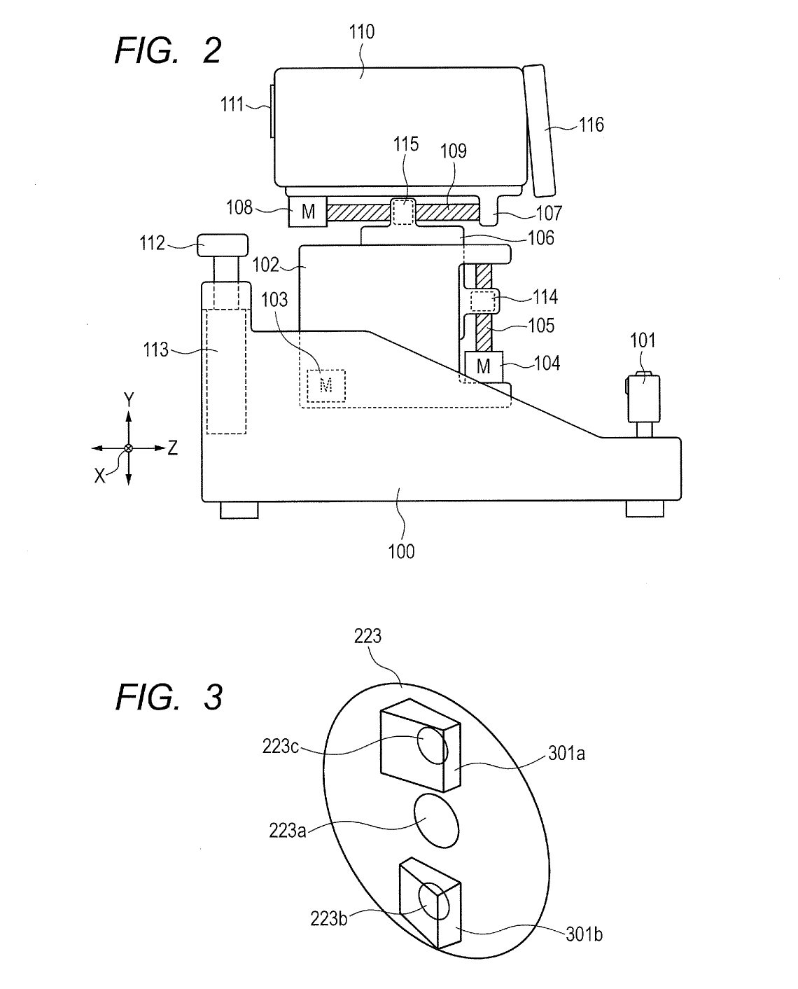

FIG. 2 illustrates a structural diagram of an eye refractometer that is an example of an ophthalmologic apparatus according to the present invention.

(Apparatus Structure)

In FIG. 2, at an end portion of a measurement unit 110 (described later in detail) on a subject side, there is disposed a light source unit 111 including a light source (described later in detail) for performing alignment. The light source unit 111 may further include a light source for measuring a corneal curvature or the like. In addition, a joystick 101 is disposed to a base 100, and the joystick 101 is an operating member for aligning the measurement unit 110 to an eye to be inspected. When the alignment is performed for measurement, the joystick is tilted for performing the alignment.

When measuring refractive power, the subject puts his or her chin on a chin rest 112 and presses his or her forehead to a forehead rest of a face rest frame (not shown) fixed to the base 100 so that a position of the eye to be inspected can be fixed. Note that, a position of the chin rest 112 can be adjusted in a Y axis direction by a chin rest drive mechanism 113 in accordance with a size of the subject's face. At an end portion of the measurement unit 110 on an inspector side, there is disposed an LCD monitor 116 as a display unit for observing the eye to be inspected, and the LCD monitor 116 can display measurement results or the like.

(Anterior Eye Part Observation and Alignment Using Index)

(1) Alignment Optical System

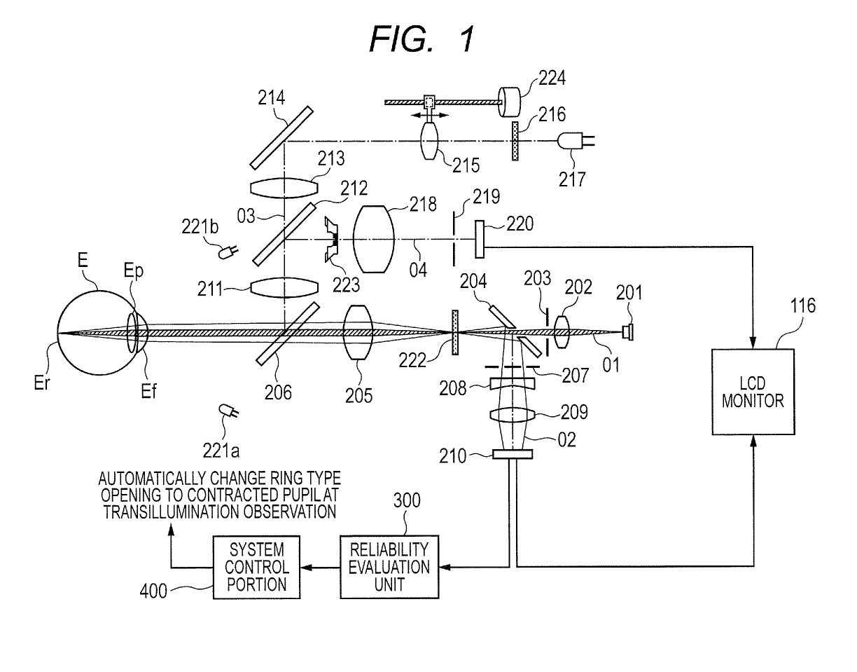

In FIG. 1 concerning the measurement unit 110, in a reflection direction of a dichroic mirror 206, there is disposed an alignment light-receiving optical system that is shared for anterior eye part observation (rough alignment) of the eye to be inspected and alignment detection (fine alignment) using an index. In other words, on an optical path 04 in the reflection direction of a dichroic mirror 212, there are disposed an alignment prism stop 223 (inserted in and removed from the optical path by a solenoid (not shown)), a lens 218, and an image sensor 220 in this order.

In diagonal front of the anterior eye part of the eye to be inspected, anterior eye part illuminating light sources 221a and 221b having a wavelength of approximately 780 nm are disposed. A light beam of an anterior eye part image of the eye to be inspected illuminated by the anterior eye part illuminating light sources 221a and 221b forms an image on a light-receiving sensor surface of the image sensor 220 as follows. Specifically, the light beam forms the image on the light-receiving sensor surface of the image sensor 220 via the dichroic mirror 206, a lens 211, the dichroic mirror 212, and a center opening 223a of the alignment prism stop (FIG. 3).

The anterior eye part image of an eye to be inspected E photographed by the image sensor 220 is stored in a memory (not shown), and a pupil of the eye to be inspected E and a corneal reflection image described below are extracted from the image stored in the memory (not shown) for performing the alignment detection. Note that, the anterior eye part image of the eye to be inspected E photographed by the image sensor 220 is combined with character or figure data and can be displayed on the LCD monitor 116.

A measurement light source 201 for measuring eye refractive power also works as the light source for the alignment detection. When the alignment is performed, a translucent diffusion plate 222 is inserted in the optical path by a diffusion plate insertion/removal solenoid (not shown). The position at which the diffusion plate 222 is inserted is a primary image forming position by a projection lens 202 of the measurement light source 201 and a focal position of a lens 205. Thus, an image of the measurement light source 201 is temporarily formed on the diffusion plate 222 to be a secondary light source, which is projected from the lens 205 toward the eye to be inspected E as a thick collimated light beam.

This collimated light beam is reflected by a cornea Ef of the eye to be inspected to form a bright spot image, and the light beam is reflected partially by the dichroic mirror 206 again. Then, the light beam is reflected by the dichroic mirror 212 via the lens 211, passes through the opening 223a of the alignment prism stop and alignment prisms 301a and 301b illustrated in FIG. 3, and is condensed by the lens 218 to form an image on the image sensor 220. The light beam having a wavelength of 780 nm of the anterior eye part illuminating light sources 221a and 221b or longer passes through the center opening 223a of the alignment prism stop 223.

Therefore, the reflected light beam of the anterior eye part image illuminated by the anterior eye part illuminating light sources 221a and 221b propagates in an observation optical system similarly to the path of the reflected light beam of the cornea Ef and forms an image on the image sensor 220 by the imaging lens 218 via the opening 223a of the alignment prism stop 223. In addition, the light beam after passing through the alignment prism 301a is refracted in a downward direction, while the light beam after passing through the alignment prism 301b is refracted in an upward direction. Based on a positional relationship between the light beams after passing through the stops, the alignment between the eye to be inspected E and the apparatus can be performed.

By insertion and removal of the alignment prism stop 223, it is possible to perform the alignment when the alignment prism stop 223 is on the optical path 04, and to perform anterior eye part observation or transillumination observation (described later in detail) when the alignment prism stop 223 is removed from the optical path. Here, FIG. 3 illustrates a shape of the alignment prism stop 223. Three openings 223a, 223b, and 223c are formed in the disk-like diaphragm plate, and the alignment prisms 301a and 301b for transmitting only the light beam having a wavelength near 880 nm are respectively bonded to the openings 223c and 223b of both sides on the dichroic mirror 212 side.

When the alignment is performed, a cornea bright spot formed by the light beam reflected by a cornea Ef is split by the openings 223a, 223b, and 223c of the alignment prism stop 223 and the prisms 301a and 301b. Then, the split bright spots are photographed by the image sensor 220 as index images Ta, Tb, and Tc together with the anterior eye part image of the eye to be inspected E illuminated by the anterior eye part illuminating light sources 221a and 221b and bright spot images 221a′ and 221b′ of the anterior eye part illuminating light sources 221a and 221b. The light beam after passing through the alignment prism 301a of FIG. 3 is refracted in a left direction, while the light beam after passing through the alignment prism 301b is refracted in a right direction, and hence the three bright spots Ta, Tb, and Tc can be acquired.

In addition, when the three bright spots Ta, Tb, and Tc are detected, a system control portion 400 controls a motor driving circuit (not shown) to drive the measurement unit 110 in up, down, left, and right directions so that the center bright spot Tc first coincides with the central direction. Next, the system control portion 400 drives the measurement unit 110 in the front and back direction so that the bright spots Ta and Tb are aligned to the bright spot Tc in the horizontal direction (lateral direction, or left and right direction). Then, the alignment is completed in a state where the three cornea bright spots Ta, Tb, and Tc are aligned in the horizontal direction.

Note that, the alignment prism stop 223 is disposed so that the stops are aligned in the vertical direction on the optical path as illustrated in FIG. 3, but the stops may be aligned in the lateral direction. In this case, the light beam is refracted by the respective prisms in the upward and downward directions. When the measurement unit is aligned in the front and back direction, the three bright spots are aligned in the up and down direction.

(2) Movement of Apparatus

In FIG. 2, a frame 102 can move in the left and right direction (hereinafter referred to as an X axis direction) with respect to the base 100. A drive mechanism in the X axis direction includes an X axis drive motor 103 fixed onto the base 100, a feed screw (not shown) connected to an output shaft of the motor, and a nut (not shown) fixed to the frame 102 in a movable manner in the X axis direction on the feed screw. When the motor 103 rotates, the frame 102 moves in the X axis direction through the intermediation of the feed screw and the nut.

A frame 106 can move in the up and down direction (hereinafter referred to as the Y axis direction) with respect to the frame 102. A drive mechanism in the Y axis direction includes a Y axis drive motor 104 fixed onto the frame 102, a feed screw 105 connected to an output shaft of the motor, and a nut 114 fixed to the frame 106 in a movable manner in the Y axis direction on the feed screw. When the motor 104 rotates, the frame 106 moves in the Y axis direction through the intermediation of the feed screw and the nut.

A frame 107 can move in the front and back direction (hereinafter referred to as a Z axis direction) with respect to the frame 106. A drive mechanism in the Z axis direction includes a Z axis drive motor 108 fixed onto the frame 107, a feed screw 109 connected to an output shaft of the motor, and a nut 115 fixed to the frame 106 in a movable manner in the Z axis direction on the feed screw. When the motor 108 rotates, the frame 107 moves in the Z axis direction through the intermediation of the feed screw 109 and the nut 115. The measurement unit 110 for performing the measurement is fixed onto the frame 107.

(Measurement of Eye Refractive Power Information)

(1) General Structure and Measurement Principle of Measurement Unit

FIG. 1 is an optical system layout diagram inside the measurement unit 110. In an eye refractive power information measurement mode using the measurement unit 110 as a specific information acquiring unit for acquiring (measuring) eye refractive power as specific information of the eye to be inspected, the light sources are turned ON and OFF as follows. Specifically, in the eye refractive power information measurement mode, the eye refractive power measuring light source 201 emitting light having a wavelength of 880 nm is turned ON, while the anterior eye part illuminating light sources 221a and 221b are turned OFF, and the diffusion plate 222 is disposed outside the optical path. Note that, the alignment prism stop 223 may be in the optical path or may be outside the optical path.

On an optical path 01 from the eye refractive power measuring light source 201 emitting light having a wavelength of 880 nm to the eye to be inspected E, there are disposed the lens 202, a stop 203 substantially conjugate with a pupil Ep of the eye to be inspected E, a perforated mirror 204, and the lens 205 in this order. Further, there is disposed the dichroic mirror 206 that totally reflects infrared and visible light having a wavelength of 880 nm or shorter and partially reflects a light beam having a wavelength of 880 nm or longer from the eye to be inspected E side.

On an optical path 02 in the reflection direction of the perforated mirror 204, there are disposed a stop 207 having a ring-type slit substantially conjugate with the pupil Ep, a light beam split prism 208, a lens 209, and an image sensor 210 in this order. The above-mentioned optical system is used for measuring eye refractive power. The light beam emitted from the measurement light source 201 is restricted by the stop 203 and forms a primary image by the lens 202 before the lens 205, passes through the lens 205 and the dichroic mirror 206, and is projected to the pupil center of the eye to be inspected E.

Reflected light of the projected light beam passes through the pupil center and enters the lens 205 again. The incident light beam is reflected by the periphery of the perforated mirror 204 after passing through the lens 205. The reflected light beam is separated by pupil separation by the stop 207 substantially conjugate with the pupil Ep of the eye to be inspected and by the light beam split prism 208, and is projected to a light-receiving surface of the image sensor 210 as a ring image. This ring image can be displayed on the LCD monitor 116 together with an eye refractive power value, but it is possible to adopt a structure in which the ring image is not displayed on the LCD monitor 116.

When the eye to be inspected E is an emmetropic eye, the ring image projected onto the light-receiving surface of the image sensor 210 becomes a predetermined circle. The circle becomes smaller in a short-sighted eye than in the emmetropic eye, while the circle becomes larger in a long-sighted eye than in the emmetropic eye. When the eye to be inspected E has astigmatism, the ring image becomes an ellipse in which an angle between a horizontal axis and the ellipse is an astigmatism axis angle. Based on a coefficient of this ellipse, the refractive power is determined.

Here, as illustrated in FIG. 5, a diaphragm plate 311 works as a first member forming a ring type opening 207a for a normal pupil diameter that is a first opening for measurement of the normal pupil diameter as the stop 207. In addition, the diaphragm plate 311 also works as a second member forming a ring type opening 207b for a small pupil diameter that is a second opening having smaller inner and outer diameters than the normal pupil diameter 207a. In other words, a radius of the ring type opening 207b for the small pupil diameter from the optical axis (corresponding to the inner diameter of the ring type opening 207b for the small pupil diameter) is set to be smaller than a radius of the ring type opening 207a for the normal pupil diameter from the optical axis (corresponding to the inner diameter of the ring type opening 207a for the normal pupil diameter).

The diaphragm plate 311 is rotated about an axis 304 by drive of an actuator 302 and has multiple stop positions. At each of the stop positions, the ring type opening 207a for the normal pupil diameter or the ring type opening 207b for the small pupil diameter, which can be inserted in or removed from the optical path, is disposed on the optical axis.

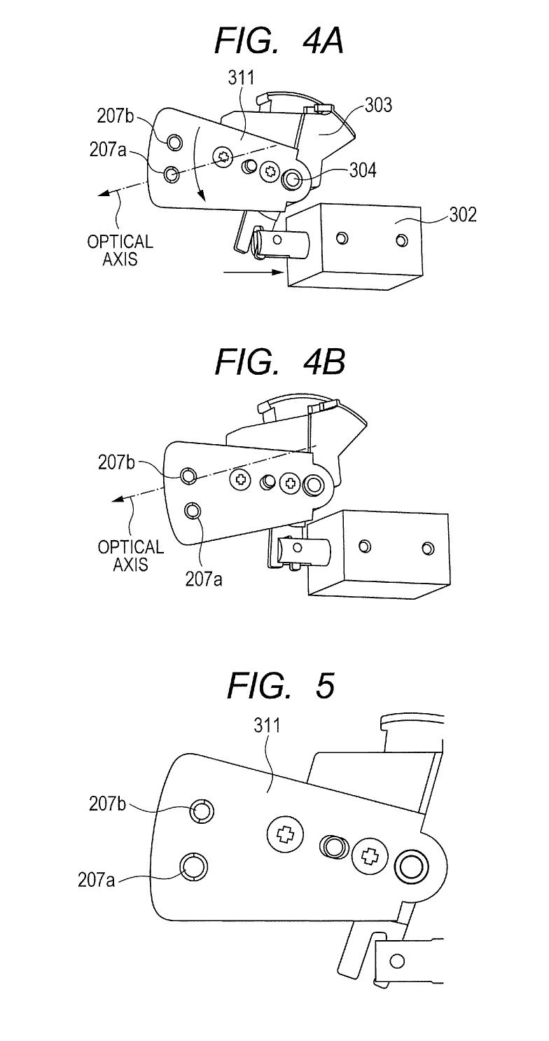

In an initial state, as illustrated in FIG. 4A, the ring type opening 207a for the normal pupil diameter is disposed substantially on the optical axis. When an actuator drive signal is applied from a main body control unit, the actuator 302 is driven so that a lever 303 is turned in a counterclockwise direction and is stopped by a stopper (not shown). In this case, the ring type opening 207a for the normal pupil diameter is removed from the optical path, and the ring type opening 207b for the small pupil diameter formed in the diaphragm plate 311 is inserted in the optical path and is disposed substantially on the optical axis (FIG. 4B).

This embodiment describes an example in which the diaphragm plate 311 has the two ring type openings having different inner diameters and different outer diameters, but it is possible that the diaphragm plate has multiple ring type openings having different inner diameters only. Note that, any structure can be adopted as long as a small pupil can be photographed when the small pupil is photographed. For instance, it is possible to adopt the structure as described in Japanese Patent Application Laid-Open No. 2004-180708, in which a shielding member is inserted when a small pupil is photographed. In addition, it is preferred that the display unit display a display form indicating a change to the stop for a small pupil. Thus, a user can easily recognize visually the current state of the apparatus, and hence an operation error can be prevented.

(2) Fixation Target

On the other hand, a fixation target projection optical system is disposed in the reflection direction of the dichroic mirror 206. On an optical path 03 of the fixation target projection optical system, there are disposed the lens 211, the dichroic mirror 212, a lens 213, a reflection mirror 214, a lens 215, a fixation target 216, and a fixation target illumination light source 217 in this order.

A light beam projected from the fixation target illumination light source 217 that is turned ON for fixation guide illuminates the fixation target 216 from the backside, and the light is projected to a fundus Er of the eye to be inspected E via the lens 215, the reflection mirror 214, the lens 213, the dichroic mirror 212, and the lens 211. Note that, the lens 215 can be moved in the optical axis direction by a fixation guide motor 224 so as to perform diopter guide of the eye to be inspected E and to realize a fogged state.

Here, the fixation target 216 is disposed at a predetermined reference position so as to perform preliminary measurement (first measurement). Based on the determined eye refractive power value, the lens 215 is moved to a position corresponding to the refractive power value by driving a fixation target guide motor (not shown) via the motor driving circuit. Thus, the fixation target 216 is displayed on the eye to be inspected E at a refractivity corresponding to a refractivity of the eye to be inspected E. After that, the lens 215 is moved to the far side by a predetermined amount so that the fixation target 216 is fogged, and the measurement light source 201 is turned ON again so as to measure refractive power. In this way, measurement of the refractive power, fogging by the fixation target 216, and measurement of refractive power are repeated so as to acquire a final measured value in which the refractive power is stabilized.

(Transillumination Observation Mode)

In a transillumination observation mode in which a pupil area (transillumination image) illuminated by the reflected light beam from the fundus of the eye to be inspected is observed as a moving image, the light sources are turned ON and OFF as follows. Specifically, the measurement light source 201 is turned ON, while the anterior eye part illuminating light sources 221a and 221b are turned OFF. Then, the diffusion plate 222 and the alignment prism stop 223 are removed from the optical path. In other words, the light beam, which is projected from the measurement light source 201 to the fundus Er and is reflected by the fundus Er, illuminates the pupil area, and a part of the light beam in the pupil area is reflected by the dichroic mirror 206, and is reflected by the dichroic mirror 212 via the lens 211.

Then, the pupil area is projected to the image sensor 220 by the lens 218. The pupil area projected to the image sensor 220 as a transillumination image acquiring unit that acquires the transillumination image is displayed on the LCD monitor 116, and it is possible to observe whether or not the pupil area has an opacified part.

In the transillumination observation mode, as described later, the ring type opening 207a for the normal pupil diameter is changed to the ring type opening 207b for the small pupil diameter, light intensity of the measurement light source 201 is increased, and a photography gain of the image sensors 220 and 210 such as a CCD is increased, so as to increase light-receiving sensitivity. Such switching and change are automatically performed by the system control portion 400 (FIG. 1) in synchronization with a start signal for the display unit to display the reflected light beam from the pupil area of the eye to be inspected by transillumination observation or the fundus of the eye to be inspected that is photographed in the eye refractive power information measurement mode. Note that, the turning ON and OFF and light intensity change of the measuring light source 201, the anterior eye part illuminating light sources 221a and 221b, and the fixation target light source 217 are also performed by using the system control portion 400 (FIG. 1).

The change from an eye refractive power information detection mode by an eye refractive power information detection unit to the transillumination observation mode by a transillumination observation unit is automatically performed when a predetermined condition is not satisfied as described below in detail. In this case, changing of the ring type opening, light intensity increase of the measurement light source 201, removal of the diffusion plate 222 and the alignment prism stop 223 outside the optical path, turning OFF of the anterior eye part illuminating light sources 221a and 221b are specifically performed as the automatic change to the transillumination observation mode.

In the transillumination observation mode in which the transillumination observation is performed, the reflected light of the light beam projected to the fundus of the eye to be inspected is separated by pupil separation by the stop 207 substantially conjugate with the pupil Ep of the eye to be inspected and by the light beam split prism 208, and can be photographed as a ring image by the image sensor 210 on the light-receiving surface. Then, based on a state of the ring image as the photographed reflected light beam, the aligned state of the apparatus with respect to the eye to be inspected can be changed.

(Entire System Control)

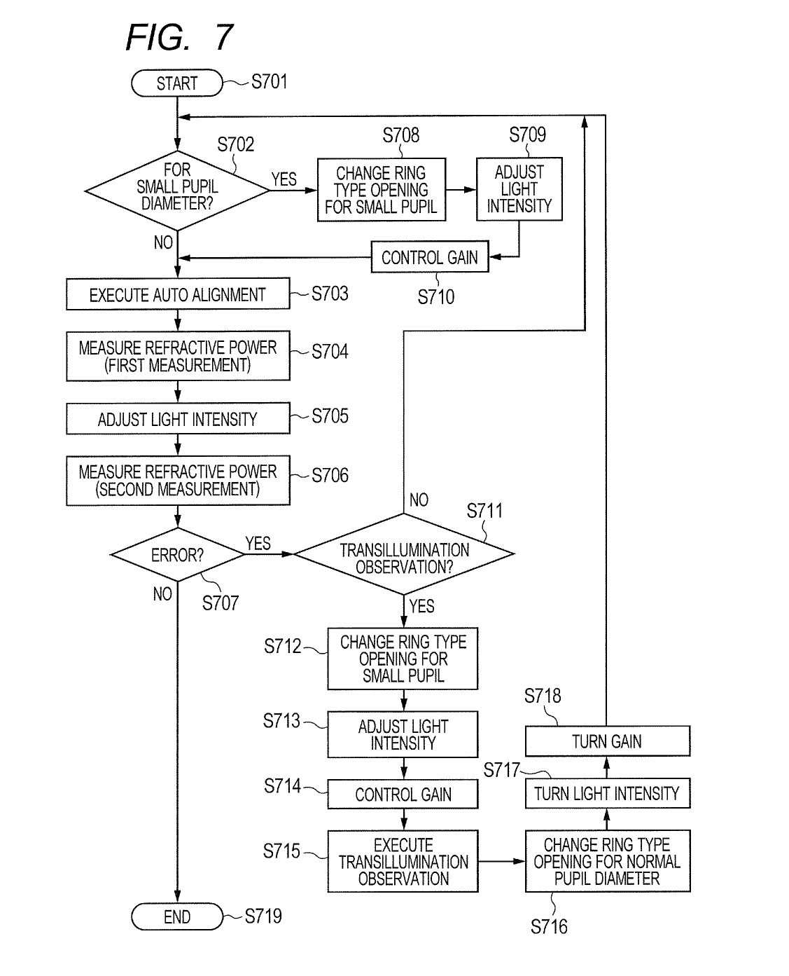

Next, an actual measurement flow (FIG. 7) is described. When the measurement is started (S701), the system control portion (not shown) starts automatic alignment so as to perform the alignment of the measurement unit. In this case, as to the ring type opening 207, the ring type opening 207a for the normal pupil diameter measurement is substantially on the optical axis (FIG. 4A), but the inspector may select the ring type opening 207b for the small pupil diameter to be substantially on the optical axis (S702). When the alignment is completed (S703), in order to measure the eye refractive power (first measurement), the measurement light source 201 projects the measuring beam to the fundus Er of the eye to be inspected E. As the light intensity in this case, a value adjusted in the manufacturing process is used.

The reflected light from the fundus Er is received by the image sensor 210 as a ring image via the ring type opening 207 disposed at a position substantially conjugate with the pupil Ep, and the eye refractive power of the eye to be inspected E is calculated from the ring image. Thus, the first measurement is completed (S704). Here, light intensity adjustment is performed for determining light intensity in subsequent second measurement from the brightness of the ring image obtained in the first measurement (S705). For instance, when the brightness of the obtained ring image is lower than a predetermined value, the light intensity is increased. On the contrary, when the brightness is higher than the predetermined value, the light intensity is decreased. These are different depending on the eye to be inspected, and the light intensity determined here becomes an initial value for measuring the subject.

Next, the eye refractive power is measured (second measurement) (S706). Based on the eye refractive power value obtained by the first measurement, the lens 215 is moved to a position corresponding to the refractive power value by the fixation target guide motor 224, and the fixation target 216 is displayed on the eye to be inspected E at a refractivity corresponding to the refractivity of the eye to be inspected E.

After that, the lens 215 is moved to the far side by a predetermined amount so that the fixation target 216 is fogged, and the measurement light source 201 is turned ON again so as to measure the refractive power. In this way, measurement of the refractive power, fogging by the fixation target 216, and measurement of the refractive power are repeated so as to acquire a final measured value in which the refractive power is stabilized. Thus, the second measurement is finished. In other words, when no measurement error occurs, a result of the measurement (a result of acquiring specific information of the eye to be inspected) is displayed on the LCD monitor 116, and the measurement is finished.

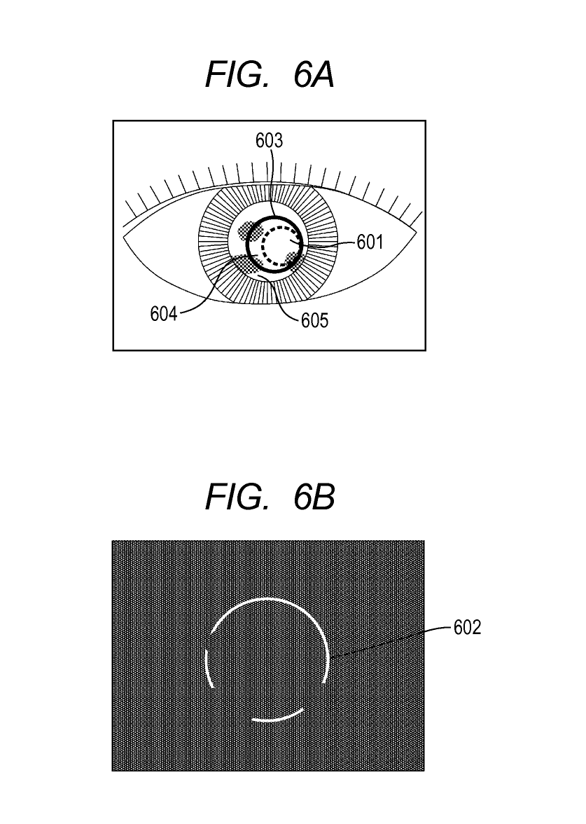

However, when the crystalline lens of the eye to be inspected has opacity, and when the pupil center transmitting the light beam projected from the measurement light source 201 is agreed with the opacified part, the projected light beam does not reach the fundus Er. Then, the image sensor 210 cannot photograph the reflected light, and hence the measurement itself cannot be performed. In addition, in a case of an eye to be inspected 601 having opacity in a part as illustrated in FIG. 6A, even when the projected light beam reaches the fundus Er, the reflected light is partially blocked by the opacity. In other words, as illustrated in FIG. 6B, a part of a ring image 602 for calculating the refractive power drops off or is blurred so that only a low reliability result of the measurement is obtained.

Therefore, a reliability evaluation unit 300 (FIG. 1) detects a case where the ring image for calculating the eye refractive power cannot be obtained or a case where a correct result of the measurement cannot be obtained because a part of the ring image drops off or is blurred in the eye refractive power measurement mode (S707). When the reliability evaluation unit 300 determines there is opacity so that transillumination observation is necessary, the eye refractive power measurement mode is automatically changed to the transillumination observation mode (S711). Note that, it is possible to manually change from the eye refractive power measurement mode to the transillumination observation mode. In addition, it is possible to adopt a structure in which, without using the reliability evaluation unit 300, the inspector can select to change from the eye refractive power measurement mode to the transillumination observation mode as necessary.

In any case, when the mode is changed to the transillumination observation mode, a signal for displaying a transillumination observation image is output. In synchronization with this, the lever 303 to which the diaphragm plate 311 having the ring type opening 207 is mounted is rotated by drive of the actuator 302. In other words, when the mode is changed to the transillumination observation mode, the ring type opening 207b for the small pupil diameter measurement is automatically disposed substantially on the optical axis (S712) (FIG. 4B).

In this case, the measurement light source is turned ON so that the light beam is projected to the fundus. In this state, in synchronization with the signal for changing to the ring type opening for the small pupil diameter, the light intensity of the measurement light source is increased by a predetermined amount (S713), and the gain of the CCD as a light receiving unit is increased, so as to increase the light-receiving sensitivity (S714). Thus, it is possible to avoid a variation or the like of the measured value due to lack of received light intensity when changing to the ring type opening 207b for the small pupil diameter. It is possible that only one of the measurement light intensity and the light-receiving gain is synchronized with the signal for changing the ring type opening 207.

In addition, because the LCD display unit 116 displays the transillumination image and the ring image, the inspector operates the joystick 101 so as to align the measurement unit 110 to a position without opacity of the crystalline lens where the ring image does not overlap the opacified part (S715). Note that, because the ring image is photographed by the image sensor 210 for alignment, it is possible to perform the alignment by using the photographed image signal without displaying the ring image on the LCD display unit 116.

When the alignment of the measurement unit 110 is completed, the inspector presses a measurement start button so as to change from the transillumination observation mode to the measurement mode. When the measurement start button is pressed so that a signal for finishing the transillumination observation mode is output from the main body control unit, the ring type opening 207 is changed to the ring type opening 207a for the normal pupil diameter in synchronization with the signal (S716). At the same time, the measurement light intensity is reset to the value before changing to the ring type opening for the small pupil diameter (S717), and the gain of the CCD as the light receiving unit is reset to the value before changing to the ring type opening for the small pupil diameter (S718).

Note that, when the alignment of the measurement unit 110 is completed, the inspector presses the measurement start button so that the signal for finishing the transillumination observation mode is output from the main body control unit. However, as described above, it is possible to change to the measurement mode while the ring type opening 207b for the small pupil diameter measurement is maintained. In other words, when changing from the transillumination observation mode to the eye refractive power information measurement mode using an eye refractive power information measuring unit, it is possible to perform the measurement of eye refractive power without changing to the ring type opening 207a for the normal pupil diameter while the ring type opening 207b for the small pupil diameter measurement is maintained.

When the crystalline lens has no opacity as a result of the transillumination observation, the inspector presses the measurement start button so as to perform the measurement in an automatic measurement mode again without performing the alignment adjustment of the measurement unit. In this case too, as described above, when the signal for finishing the transillumination observation mode is output from the main body control unit, the ring type opening is changed to the ring type opening 207a for the normal pupil diameter in synchronization with the signal.

In this embodiment, opacity of the crystalline lens is automatically determined, and the measurement mode is automatically changed to the transillumination observation mode. However, also in a case where the measurement mode is manually changed to the transillumination observation mode, the ring type opening is automatically changed similarly in synchronization with a signal for starting the transillumination observation mode (S708). According to this embodiment, when changing to the transillumination observation mode, it is possible to automatically change the ring type opening conjugate with the pupil of the eye to be inspected to the opening having a smaller radius from the optical axis, and hence it is possible to easily and quickly find a part of the crystalline lens without opacity where the eye refractive power can be measured.

Further, when changing to the eye refractive power information measurement mode, it is possible to automatically change the ring type opening conjugate with the pupil of the eye to be inspected to the opening having a larger radius from the optical axis, and hence it is possible to easily and quickly perform the measurement of the eye refractive power in a part of the crystalline lens without opacity where the eye refractive power can be measured.

The embodiment of the present invention is described above, but it is possible to variously combine or modify the technical matters described above within the scope of the present invention.

### Modified Example 1

In the embodiment described above, the light beam is projected from the center of the pupil of the eye to be inspected to the fundus of the eye to be inspected, and the reflected light beam from the fundus of the eye to be inspected is received through the periphery of the pupil of the eye to be inspected via the ring type openings 207a and 207b in the reflection optical path from the fundus of the eye to be inspected. However, it is possible to adopt the opposite structure. Specifically, it is possible to adopt the following structure. In the projection optical path to the fundus of the eye to be inspected, the light beam is projected to the fundus of the eye to be inspected through the periphery of the pupil of the eye to be inspected via the ring type opening disposed at the position conjugate with the pupil of the eye to be inspected, and the reflected light beam from the fundus of the eye to be inspected is received through the center part of the pupil of the eye to be inspected.

In addition, instead of disposing the ring type opening 207a as the first opening and the ring type opening 207b as the second opening at the same position in the optical path like the embodiment described above, it is possible to dispose the ring type openings 207a and 207b at positions conjugate optically with each other.

In addition, instead of the ring type opening 207a as the first opening and the ring type opening 207b as the second opening, it is possible to adopt a structure having an opening in at least three meridional directions. In this case, when automatically or manually changing to the transillumination observation mode by the transillumination observation unit, the opening in at least three meridional directions (first opening) is automatically changed to a second opening having a smaller radius from the optical axis in at least three meridional directions. In this case too, the radius of the opening from the optical axis when the second opening is projected to the pupil of the eye to be inspected is set smaller than the radius of the opening from the optical axis when the first opening is projected to the pupil of the eye to be inspected.

Then, with respect to the opening as the first opening, the opening as the second opening may be the same meridional directions or may be changeable to different meridional directions. In other words, it is possible to set the opening as the first opening to three meridional directions at 0 degrees, 120 degrees, and 240 degrees, and to set the opening as the second opening to different three meridional directions (for example, at 45 degrees, 165 degrees, and 285 degrees) so as to avoid a part of the crystalline lens with opacity.

### Modified Example 2

In the embodiment described above, when changing from the transillumination observation mode to the eye refractive power information measurement mode, the opening is reset to the ring type opening 207a for the normal pupil diameter for measuring the eye refractive power. However, it is possible to measure the eye refractive power while maintaining the ring type opening 207b for the small pupil diameter.

### Modified Example 3

In the embodiment described above, the ring type opening 207a for the normal pupil diameter is disposed on the optical axis in the initial state. However, it is possible to dispose the ring type opening 207b for the small pupil diameter on the optical axis in the initial state by the inspector's selection, for example. In this case, when the mode is changed to the eye refractive power information measurement mode from the initial state, the opening is changed to the ring type opening 207a for the normal pupil diameter.

After that, when the eye refractive power information measurement mode is automatically or manually changed to the transillumination observation mode, the ring type opening 207a for the normal pupil diameter is automatically changed to the ring type opening 207b for the small pupil diameter similarly to the above description.

Further, when the transillumination observation mode is automatically or manually changed to the eye refractive power information measurement mode, the ring type opening 207b for the small pupil diameter is automatically changed to the ring type opening 207a for the normal pupil diameter similarly to the above description.

### Modified Example 4

Note that, the embodiment describes an eye refractive power measuring apparatus, but it is possible that the apparatus also has other functions such as a cornea shape measuring function. In addition, the present invention can be applied similarly to other ophthalmologic apparatus such as a fundus camera, a fundus blood flow meter, or a fundus tomographic image pickup apparatus (OCT) using optical interference of a near infrared laser. In addition, the image sensor may be any one of an area sensor and a line sensor. In addition, the eye refractive power measuring apparatus may be one using a measurement principle other than that described above in this embodiment.

### Other Embodiments

In addition, the present invention is further applied to an ophthalmologic control method, which includes a specific information acquiring step of acquiring specific information of an eye to be inspected by a first opening, a transillumination image acquiring step of acquiring a transillumination image of the eye to be inspected, and a control step of changing the first opening to a second opening smaller than the first opening when acquiring the transillumination image.

Further, the method can be also realized by an ophthalmologic control program in which the following processing is executed. Specifically, software (program) for realizing functions of the embodiment described above is supplied to a system or an apparatus via a network or various storage media, and a computer (CPU, MPU, or the like) of the system or the apparatus reads and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-279585, filed Dec. 21, 2011, which is hereby incorporated by reference herein in its entirety.

What is claimed is:

1. An ophthalmologic apparatus, comprising:
a specific information acquiring unit which acquires specific information of an eye to be inspected through a first opening;
a transillumination image acquiring unit which acquires a transillumination image of the eye to be inspected; and
a control unit which changes the first opening to a second opening smaller than the first opening when acquiring the transillumination image.

2. An ophthalmologic apparatus according to claim 1, wherein the control unit changes the second opening to the first opening when acquiring the specific information.

3. An ophthalmologic apparatus according to claim 1, further comprising:
a first member forming the first opening, which is disposed in an optical path of the specific information acquiring unit in an insertable and removable manner; and
a second member forming the second opening, which is disposed in the optical path of the specific information acquiring unit in an insertable and removable manner,
wherein the control unit removes the first member from the optical path and inserts the second member into the optical path when acquiring the transillumination image.

4. An ophthalmologic apparatus according to claim 1, wherein, when the specific information does not satisfy a predetermined condition, the control unit controls the transillumination image acquiring unit to acquire the transillumination image as a moving image.

5. An ophthalmologic apparatus according to claim 4, wherein, when the specific information satisfies the predetermined condition, the control unit does not acquire the transillumination image as the moving image but controls a display unit to display an acquired result and finishes acquiring of the specific information of the eye to be inspected.

6. An ophthalmologic apparatus, comprising:
an eye refractive power information measuring unit which projects a light beam to a fundus of an eye to be inspected and photographs a reflected light beam from the fundus of the eye to be inspected so as to measure eye refractive power information, the eye refractive power information measuring unit including a first opening having an opening in at least three meridional directions at a position conjugate with a pupil of the eye to be inspected in one of a reflection optical path from the fundus of the eye to be inspected and a projection optical path to the fundus of the eye to be inspected; and
a transillumination observation unit which observes a pupil area of the eye to be inspected illuminated by the reflected light beam from the fundus of the eye to be inspected, wherein
in a transillumination observation mode for performing transillumination observation, the ophthalmologic apparatus is capable of photographing the reflected light beam from the fundus of the eye to be inspected in at least three meridional directions through a second opening having an opening in the at least three meridional directions at the position conjugate with the pupil of the eye to be inspected in the one of the reflection optical path from the fundus of the eye to be inspected and the projection optical path to the fundus of the eye to be inspected, and is capable of changing an aligned state of the ophthalmologic apparatus with respect to the eye to be inspected based on a state of the photographed reflected light beam;
the ophthalmologic apparatus further comprises a control unit which automatically changes the first opening in an eye refractive power information measurement mode to the second opening in the transillumination observation mode when changing from the eye refractive power information measurement mode using the eye refractive power information measuring unit to the transillumination observation mode; and
a radius of the opening from an optical axis when the second opening is projected to the pupil of the eye to be inspected is set smaller than a radius of the opening from the optical axis when the first opening is projected to the pupil of the eye to be inspected.

7. An ophthalmologic apparatus, comprising:
an eye refractive power information measuring unit which projects a light beam to a fundus of an eye to be inspected and photographs a reflected light beam from the fundus of the eye to be inspected so as to measure eye refractive power information, the an eye refractive power information measuring unit including a first opening having an opening in at least three meridional directions at a position conjugate with a pupil of the eye to be inspected in one of a reflection optical path from the fundus of the eye to be inspected and a projection optical path to the fundus of the eye to be inspected; and
a transillumination observation unit which observes a pupil area of the eye to be inspected illuminated by the reflected light beam from the fundus of the eye to be inspected, wherein:
in a transillumination observation mode for performing transillumination observation, the ophthalmologic apparatus is capable of photographing the reflected light beam from the fundus of the eye to be inspected in at least three meridional directions through a second opening having an opening in the at least three meridional directions at the position conjugate with the pupil of the eye to be inspected in the one of the reflection optical path from the fundus of the eye to be inspected and the projection optical path to the fundus of the eye to be inspected, and is capable of changing an aligned state of the ophthalmologic apparatus with respect to the eye to be inspected based on a state of the photographed reflected light beam;
the ophthalmologic apparatus further comprises a control unit which automatically changes the second opening in the transillumination observation mode to the first opening in an eye refractive power information measurement mode when changing from the transillumination observation mode to the eye refractive power information measurement mode using the eye refractive power information measuring unit; and
a radius of the opening from an optical axis when the first opening is projected to the pupil of the eye to be inspected is set larger than a radius of the opening from the optical axis when the second opening is projected to the pupil of the eye to be inspected.

8. An ophthalmologic apparatus according to claim 6, wherein the first opening and the second opening each have a ring type opening as the opening.

9. An ophthalmologic apparatus according to claim 6, wherein the control unit changes one of the first opening and the second opening in synchronization with a start signal for a display unit to display one of a reflected light beam from the pupil area of the eye to be inspected that is observed by the transillumination observation in the transillumination observation mode and the reflected light beam from the fundus of the eye to be inspected that is photographed in the eye refractive power information measurement mode.

10. An ophthalmologic apparatus according to claim 9, wherein a photography gain of an image pickup unit for photographing the reflected light beam from the fundus of the eye to be inspected is changed in synchronization with the start signal.

11. An ophthalmologic apparatus according to claim 9, wherein light intensity of the light beam projected to the fundus of the eye to be inspected is changed in synchronization with the start signal.

12. An ophthalmologic control method, comprising:
a specific information acquiring step of acquiring specific information of an eye to be inspected through a first opening;
a transillumination image acquiring step of acquiring a transillumination image of the eye to be inspected; and
a control step of changing the first opening to a second opening smaller than the first opening when acquiring the transillumination image.

13. An ophthalmologic control program for causing a computer to perform all the steps of the ophthalmologic control method according to claim 12.

14. An ophthalmologic apparatus according to claim 7, wherein the first opening and the second opening each have a ring type opening as the opening.

15. An ophthalmologic apparatus according to claim 7, wherein the control unit changes one of the first opening and the second opening in synchronization with a start signal for a display unit to display one of a reflected light beam from the pupil area of the eye to be inspected that is observed by the transillumination observation in the transillumination observation mode and the reflected light beam from the fundus of the eye to be inspected that is photographed in the eye refractive power information measurement mode.
